(19) United States
(12) Patent Application Publication
(10) Pub. No.: US 2024/0002764 A1
(43) Pub. Date: Jan. 4, 2024

(54) Microfluidic flow cell and system for analyzing or diagnosing biofilms and cell cultures, and the use thereof

(71) Applicant: Johannes Gutenberg-Universität Mainz, Mainz (DE)

(72) Inventors: Ralf Heermann, Frankfurt am Main (DE); Athanasios Gazanis, Wilhermsdorf (DE)

(21) Appl. No.: 18/251,818

(22) PCT Filed: Nov. 22, 2021
(86) PCT No.: PCT/EP2021/082439

§ 371 Date: May 4, 2023

(30) Foreign Application Priority Data
Nov. 23, 2020 (DE) 102020130870.1

(51) Int. Cl. C12M 3/06; C12M 1/12; C12M 1/34; B01L 3/00

(52) U.S. Cl. CPC C12M 23/16; C12M 25/04; C12M 41/46; C12M 41/44; C12M 41/12; B01L 3/502761; B01L 2400/082; B01L 2200/16

(57) ABSTRACT

Microfluidic flow cells for analyzing or diagnosing biofilms and cell cultures. The microfluidic flow cells comprise a support plate with a sample chamber formed therein, which is peripherally limited by chamber walls and a bottom, a cover plate which can be connected to the support plate in a fluid-tight manner, an inlet with an integrated inlet channel, which leads to the sample chamber via an opening, a drain with an integrated drain channel. Holding elements for fixing the support plate to a microscope stage or a holding device are attached to the front sides of the support plate. The invention further relates to systems and their use for analyzing and diagnosing biofilms and cell cultures using these microfluidic flow cells.

### TECHNICAL FIELD

The present invention relates to microfluidic flow cells and systems for analyzing and diagnosing biofilms and cell cultures. The invention further relates to the use of these microfluidic flow cells or systems for analyzing or diagnosing biofilms or cell cultures.

### STATE OF THE ART

Microfluidic systems and processes are principally known in the state of the art, as devices and processes based on microfluidic principles have been extensively established within the laboratory field. Their advantages include, among other, a lower consumption of reagents and sample materials per experimental approach while simultaneously increasing the number of experimental approaches that can be performed at the same time. There is a distinction between limited and closed microfiuidic systems, in which the support plate, the cover plate and the wall structures intended in between are permanently and not only temporarily connected to each other by clipping or jamming. The walls of the support plate, together with the cover plate and support plate, form a channel system which is restricted by corresponding inner surfaces.

Such microfluidic systems can comprise channels and cavities whose dimensions are comparable to the measurements of biological cells and tissue structures. Thus, it is possible to cultivate cells under in vivo-like conditions, for example by adjusting a defined perfusion. Under these, conditions, it is possible for the cells to receive their phenotype, which, among other things, is relevant for diagnosing, so that the results are achieved under physiological conditions, if possible. These complex cell structures can be used, for instance, to determine the toxicity, the metabolism and the mechanisms of action of active agents in the pharmaceutical industry.

A new field for the use of microfluidic flow cells is the analysis and diagnosis of biofilms, i.e., colonization of microorganisms on surfaces, for example on surfaces of drinking water systems or in pipelines of the food industry or the pharmaceutical industry. The colonization of surfaces can either occur through one type of bacteria or different types of microorganisms. In a biofilm, microorganisms are exposed to completely different environmental circumstances compared to their free-living conspecifics (Hall-Stoodley et al., 2004 Fleming et al., 2016). In a joint biofilm, microorganisms are far more resistant than free-floating individual cells. Therefore, in the medical field, much higher doses of antibiotics have to be used in order to combat biofilms of clinically relevant bacteria, as the microorganisms in the biofilm are covered in a mucoid matrix and are thus protected from the effects of antibiotics (Bryers et al., 2008; Fleming et al., 2017). However, biofilms do not only cause massive problems in the medical or hygienic sector, but also affect other branches, e.g., systems with drinking water or the naval sector, e.g., shipping industry, where biofilms develop on the hulls of the ships, exponentially increasing the ships' fuel consumption (Little et al., 2008; Niaounakis et al, 2017; de Carvalho et al., 2018). With the help of microfluidic flow cells, it is possible to comprehend and analyze these biofilms and their different appearances, as the conditions within the flow cell can be realistically replicated. For instance, in a dosed system it is possible to test the effects of medication or antimicrobial agents, which are for example applied on surfaces, material with lacquer or in drinking water pipes, on the effects of living cells in flow under several environmental circumstances.

There are microfluidic cells used for analyzing biological systems, however, these have various disadvantages, e.g., the lacking compatibility with different types or versions of microscopes, the flexibility regarding surface choice, pressure build-up and air bubble entrainment. For example, all-in-one 3D oriented microscopy chambers are known for multidimensional imaging (Alessandri, K., u.a.: All-in-one 3D printed microscopy chamber for multidimensional imaging, the UniverSlide. Sci.Rep. (2017) 7:42378). Another system is a membrane-integrated microfluidic flow cell, which is equipped with cell chambers and cover glasses (Epshteyn, A. A., u.a.: Membrane-integrated microfluidic device for high-resolution live cell imaging. Biomicrofiuidics (2811) 5(4): 046501-046501-6).

Flow cells, as they are nowadays available on the market and used in various laboratories around the world, must always meet minimum criteria to ensure concrete, realistic and reproducible results in analysis or diagnostics. For example, the microorganisms' supply with fresh growth medium or test solutions (e.g., blood serum) and the drainage of used medium must be guaranteed. For optimized analytics, it is also necessary that the flow cell is adaptable in regard to the flow rates, because microorganisms or cell cultures must be able to be cultivated within the microfluidic flow cell in a way that the cells adhering to the bottom in the biofilm are not rinsed out by excessive flow rates. Nevertheless, an optimal supply of fresh nutrients must be ensured (Gale et al., 2018). An insufficient flow rate within the microfluidic flow cell often causes, due to strong manifestations of the biomass, an uncontrolled growth, which can lead to clogging of the microfluidics.

Furthermore, existing microfluidic flow cells are not compatible with different microscopes (Pale et al., 2018; AbuZineh et al., 2018; Babic et al., 2018; Sakai et al., 2019; www.biofilms.biz; www.ibisci.com). Microscopes differ in their structure and function, hence the use of flow cells available on the market is often limited to certain types of microscopes, for example, flow cells that are transparent on only one side. For instance, those flow cells are only compatible with a CLSM (confocal laser scanning microscope) microscope, in which fluorescent samples are activated from one side of the flow cell and can only be microscopically examined on the same side. If the laser and detector are on different sides of the flow cell, when fluorescently analyzing biological samples, the microfluidic flow cell is not usable with a CLSM. Vice versa, microscopic flow cells designed for CLSM are not compatible with conventional light or fluorescence microscopes because the light or laser and detector are on different sides. In addition to these limitations, the structure of most conventional microfluidic flow cells makes them suitable for only a few conventional microscopes, i.e., those in which the objective is located above the stage. For inverted microscopes, such cells cannot be used due to the microscope's structure, since the cell already adheres to the objective without a bracket holder providing stability.

Furthermore, conventional microfluidic flow cells can only be used for analyzing or diagnosing biofilms up to a certain point because their flow rate is too low and increased pressure is built up in the sample chamber of the microfluidic flow cell, which can damage the growing biofilm. In addition, these cells leave microorganisms very little room to form biofilms, which is why, later, matured biofims can hardly be analyzed microscopically or macroscopically (www.ibisci.com, www.biofilms.biz, www.ibidi.com).

Another disadvantage of existing microfluidic flow cells is that they can only be operated in combination with a bubble trap. Otherwise, there is a risk that an air bubble, which has permeated the closed system, will destroy the entire biofilm, as there is no space for the air bubble to escape. Furthermore, it is possible that the flow breaks off, making accurate analyses impossible (Babic et al., 2018). The lack of a bubble trap often leads to the failure of an entire experimental series as the inlet or the drain of the medium within the microfluid is disturbed or prevented. In addition, the purchase of these air bubble traps is very expensive and they can only be used once.

Another problem of existing microfluidic flow cells is that it is not possible to exchange the surface in order to test other surface materials. The available cells are mostly limited to glass or polystyrene bases so that the available flow cells already contain the base for biofilm colonization. However, these bases do not depict realistic conditions of the biological systems to be examined and that is why other surfaces have to be used for the experiments or diagnostics (Pinto et al., 2019). These, again, cannot be analyzed with conventional microfluidic flow cells. Furthermore, a previously fixed base surface poses the difficulty that it is not flexible and cannot be further processed due to experiments.

Conventional cells, which are not intended for microscopy analysis, therefore have the disadvantage that, due to their structure and geometry, only prefabricated surfaces can be used (e.g., www.biofilms.biz). Therefore, it is not possible to use materials of different geometry in a flexible manner. Hence, for analyzing and diagnosing, a material to be tested would have to be adapted to a specific cell geometry, otherwise an already prefabricated material that is only suitable for a specific flow cell would have to be used. Thus, if an operator is not able to adapt his material to the corresponding conditions or even uses a material that is not prefabricated by the supplier, an analysis of biofilms within the microfluidic flow cell can only be performed under suboptimal conditions.

Conventional microfluidic flow cells mainly consist of polycarbonate (PC) or polymethyl methacrylate (PMMA) and are therefore high-priced (Wolfaardt et al., 1994; Tolker-Nielsen et al., 2014; www.plasticker.de). In addition, both types of material are very difficult to process, which is why further costs incur during processing.

### DESCRIPTION OF THE INVENTION

Against this background, it is the object of the present invention to provide a microfluidic flow cell, which eliminates the existing disadvantages in the state of art mentioned above. This object is solved by a flow cell with the characteristics according to claim 1. Preferred embodiments can be found in the subclaims.

The flow cell according to the invention addresses the existing disadvantages of existing flow cells in the state of art, and provides solutions, especially, in regard to the compatibility of microscopes, the flexibility regarding surface choice, pressure build-up and air bubble entrainment. As a result, the flow cell according to the invention becomes more flexible in its application and, if less expensive materials are used in the production, also more affordable than conventional microfluidic flow cells. Furthermore, the flow cell according to the invention is reusable, which again reduces the costs and the impact on the environment/is environmentally friendly.

The microfluidic flow cell according to the invention comprises, like already existing flow cells, a support plate with sample chamber formed therein. The sample chamber is peripherally limited by chamber walls and, depending on the embodiment, can comprise a permanently fixed or a removable bottom. On the opposite site, i.e., the top, the sample chamber is limited by a cover plate, which is connected to the support plate in a fluid-tight manner. Depending on the embodiment, the cover plate is either permanently connected to the support plate or can be flexibly removed, using corresponding holding elements. Preferably, the cover plate is held by a cover frame. This allows an easy exchange of one or more surfaces of the cover plate or of the cover plate itself. As a result, the flow cell according to the invention can be flexibly used for different applications, for example by choosing materials for the surface or their equipment. In a preferred embodiment, the cover plate is transparent, alternatively, it can also be optically opaque, depending on the embodiment.

The bottom of the flow cell is either tightly connected with the support plate or, in case of a removable version, held by a bottom frame. Thus, the surfaces of the bottom can also be exchanged flexibly. Depending on the embodiment, the bottom can be transparent or optically opaque. An inlet with an integrated inlet channel, which is preferably attached to the front side of the support plate, is also provided for medium inflow. The inlet channel leads to the sample chamber via an opening. A drain with an integrated drain channel is provided for medium discharge.

According to the invention, holding elements are now intended on the front sides of the support plate for attaching the support plate to a microscope stage or a holding device, making the flow cell compatible with a large number of different microscopes. Preferably, the holding elements are designed in a way that they can be inserted into a holding device, which, again, can be pivoted between 0° and 180° via a pivoting device. This additionally allows a vertical installation compared to conventional microfluidic flow cells, which can usually only be installed horizontally. Preferably, the flow cell according to the invention, including its holding elements, has the size of a conventional microscope stage, so that the flow cell according to the invention can be used for both normal and inverted microscopes. This characteristic eliminates the need for a so-called air bubble trap. Due to the vertical installation (i.e., in a 90° shifted arrangement compared to the horizontal installation), any possibly occurring air bubble moves upwards and can be removed, for example, by using a pump without being in contact with the sample to be analyzed. Furthermore, the two holding elements attached on the front sides enable a simple, secure, low-cost and fast attachment, for example by using rubber seals, latching lugs, clamping elements or longitudinal joints.

Previous designs of microfluidic flow cells did not allow the analysis of biofilms or the diagnostics of cell cultures at all or only to a limited extent, among other things because the existing systems build up an excessive flow pressure within the sample chamber, which damages the biofilm. Therefore, in order to reduce the flow pressure within the sample chamber, a diameter of the inlet channel of the inlet and the drain channel of the drain, which is larger than 1 mm, preferably between 1 mm and 3 mm, preferred between 1.5 mm and 2.5 mm, is intended for the flow cell according to the invention. Cells with a diameter smaller than 1 mm for the inlet channel and the outlet channel offer little space for microorganisms to develop biofilms, which is why, later, matured biofilms can hardly be analyzed microscopically and macroscopically.

It is preferably intended that the surfaces, the cover plate and/or the bottom can be removed from the support plate. In alternative embodiments, the surfaces on which the biofilm is cultivated are removable or replaceable. Preferably, the surfaces are kept in place by a bottom frame or cover frame. Preferably, the surfaces are made of materials that facilitate the formation of the biofilm. After the exchange, the surfaces can be reattached to the flow cell. The cell can be sealed with the cover plate or bottom plate. The design according to the invention enables the cultivation of biofilms between 0° and 180°, preferably between 0° and 90°. In a shifted possibility between 0° and 180°, biofilms can be cultivated on both sides of the surfaces. Of course, all angular ranges and angular values within the range between 0° and 180° are also comprised by the invention.

In a preferred embodiment, it is intended that the sample chamber is also significantly larger in design compared to existing solutions, i.e., has a larger volume, with volumes of 3.5 cm³ to 8 cm³ being preferred and a volume of between 5 and 5.5 cm³ being particularly preferred. The height of the support plate is between 6 and 12 mm, preferably about 8 mm. The high-volume design of the sample chamber contributes to a reduced pressure and thus gentle treatment of the biofilm. Furthermore, any retracted air bubbles do not get in touch with the biofilm because the chamber is sufficiently large to allow the air bubbles to escape. The height of the chamber also allows the usage in the vertical mode (i.e., compared to the horizontal mode shifted by 90°), which allows microorganisms to colonize both surfaces and thus form a biofilm. The large volume of the sample chamber enables analysis and diagnostics of samples both on a small scale (mm) and on a large scale (cm). In the process, the samples can be attached to the bottom of the sample chamber, which prevents the material from becoming blurred in the test medium.

The sample chamber of the support plate is limited on the bottom by a chamber bottom. Preferably, the cover plate and/or the bottom are removably attached to the support plate, i.e., the cover plate and/or the bottom can be completely removed from the sample chamber. Due to the removability of the cover plate and/or the bottom, the sample chamber of the microfluidic flow cell according to the invention is freely accessible, enabling the cleaning and thus reuse of the cell without further ado. In addition, the flow cell according to the invention can be quickly treated with a new medium or a washing solution. As a result, it is also possible to freely select the material and material geometry of the cover plate and the bottom to match the support plate. Concerning the height, the sample chamber, the cover plate and/or the bottom are variable and can be individually dimensioned according to the requirements or application. The cover plate and/or bottom can be attached by using one of the usual attaching methods, for example by latching, screwing together, clamping, gluing or by using a click system. To ensure the compatibility with a large number of microscopes, a length ratio of the front side to the long side including the holding elements of the support plate between 1:2.5 and 1:3.5, preferably about 1:3, is intended in a preferred embodiment.

In order to achieve the high flow rates within the sample chamber which are required for cleaning or rinsing of the cell, significantly higher flow rates are selected as when used for cultivating biofilms, where a flow rate of approximately 5 ml/hour is sufficient. In a preferred embodiment, it is therefore intended that the flow cell is equipped with a pump that is fluidically connected to the inlet and drain and provides a flow rate of at least 4 mm/sec in the sample chamber. Such high rates and the high flow volumes of the flow cell enable the sample chamber to be efficiently rinsed after usage, for example after performing an experiment, or to be thoroughly cleaned for another test run, for example to remove adhering bacteria. Furthermore, not only the diameters of the inlet or the drain of the flow cell according to the invention have been increased compared to existing solutions, but also the length of the inlet or the drain to the sample chamber has been increased. Preferably, the length of the inlet or drain is at least 3 mm, preferably between 8 mm and 10 mm, preferred about 5 mm. The extended inlets or drains prevent connecting tubes from sliding and are also suitable for very large flow rates (e.g., 100 ml/min).

In a preferred embodiment, in which the cover plate is removable, it is intended that a notch is formed on the front side of the support plate in order to attach a seal. In the alternative embodiment in which the cover plate and bottom are removable, a notch with a seal is formed on both the top and the bottom to seal the cell. The seal is preferably a silicone seal, for example a silicone pad, which externally seals the sample chamber. Preferably, the seal attached to the notch is replaceable and can be renewed if required. Alternatively, an O-ring can be used.

In a preferred embodiment, mounting holes are intended on the front side of the support plate, for example to attach a separate see-through window. Thus, the analyses or experiments to be performed and the corresponding parameters, such as the flow velocity in the sample chamber, can be monitored.

In preferred embodiments of the microfluidic flow cell according to the invention, several flow cells are connected to each other. Preferably, this is done via latching lugs, which are attached the long sides of the support plate and cooperate with corresponding receptacles for the latching lugs of adjacent flow cells. In doing so, several flow cells can be arranged next to each other in a modular manner and attached together. In an advanced embodiment, several flow cells are held by a frame which preferably consists of various frame elements, in which at least one frame element can be connected to another frame element in a pluggable manner. By removing a frame element, the frame is opened and the flow cells can be inserted into the frame via a notch. Preferably, it is intended that the individual flow cells are connected to each other via corresponding connecting devices, for example the previously mentioned latching lugs or latching receptacles. The notch of the frame cooperates with holding elements of the flow cell attached on the front sides of the support plate.

The invention further relates to a system for analyzing and diagnosing biofilms and cell cultures, comprising one or more microfluidic flow cells as described herein, and a holding device for holding one or more flow cells. The holding device is preferably designed in a way that one or more flow cells can be pivoted within an angular range of 0° to 180°, preferably 0° to 90°. The system further comprises a pump fluidly connected to the inlet and drain. Preferably, the pump is designed in a way that the required flow rate of at least 4 mm/sec in the sample chamber can be achieved, when needed.

In a preferred embodiment, the holding device is intended to be a frame having various frame elements. Preferably, the frame has a notch for receiving one or more flow cells that cooperates with the holding elements of the support plate of a flow cell.

In a preferred embodiment, the system comprises a stand with a holding device (preferably in the form of a frame) for holding one or more flow cells. Preferably, the holding device is attached in a pivotable manner via rotary joints so that the flow cell or the frame with several flow cells can be pivoted within an angular range of 0° to 180°. In a preferred embodiment, the rotary joints are motor-driven. Alternatively, instead of a rotary joint, a stepper motor can be used, which can adjust the angle electronically. Preferably, the control of the stepper motor is software-based, for example via a mobile communication device, such as a smartphone. Preferably, holding elements are intended for supporting the rotary joints attached to both sides of the holding device in order to rotate the flow cell(s) or the holding device within the angular range of 0° to 180°, In addition, a pump is intended which is fluidically connected to the inlet and drain. The holding device is preferably mounted on a stand, with supporting elements connected to the rotary joints.

In order to keep the production costs of a flow cell according to the invention as low as possible and also due to its beneficial applicability as a sample surface, at least the support plate is entirely made of polyethylene (PE). In an alternative embodiment, the use of polypropylene (PP) is also possible, since this material is autoclavable. This choice of material allows optional processing of the cell in order to adapt it to the particular experimental situation. Through the use of flexible surfaces within the sample chamber the microfluidic flow cell according to the invention becomes even more flexible in its application. The reusability of the cells and their less expensive production are further important advantages over conventional solutions.

In an advanced embodiment of the invention, the microfluidic flow cell comprises control elements in order to control the microfluidic flow cell in a semi-automatic or fully automatic way. The aim is to control the cultivation of biofilms or cell cultures on the corresponding surfaces and to perform the incubation according to a program sequence. The key component here is a microcontroller that can be used to drive one or more motor drivers, which in turn are responsible for operating drive units (e.g., stepper motors). In the process, a first stepper motor acts as a pump in order to pump the incubation medium via the inlet into the sample chamber. Another drive unit can be pivoted in order to rotate the flow cells within an angular range of 0° to 180°, preferably 0° to 90°. Via the microcontroller, it is also possible to perform a time-dependent gradual pivoting of the flow cell over the angular range. For example, it can be intended that an angle of 0° is selected during the adhesion phase. Via a temporal gradient, the plate can then be gradually rotated up to an angle of 90°. For example, this also allows air bubbles to escape, so that they do not interfere with the formation of the biofilms or cell cultures. The controller can also ensure that the medium trickles down as gently as possible onto the biofilms or cell cultures via a preset angle. The microcontroller also receives sensor-transmitted data, through which it can perform appropriate control. For example, a flow sensor is responsible for determining the flow rate, which in turn can be adjusted via the system control. A temperature sensor can ensure that a heating element is switched on as soon as the temperature in the bottom chamber falls below a predefined value. Vice versa, a fan can ensure that the temperature is regulated when it reaches a predefined threshold. The combination of heating element and fan thus enables ideal incubation conditions within the sample chamber. A timer or time control also ensures when and for how long certain process steps are carried out, for example.

The microfluidic flow cells according to the invention can be used for analyzing and diagnosing biological samples or biofilms. They enable micro- and macroscopic analyses of biological samples and facilitate the analysis of biofilms in drinking water and supply lines as well as in pipelines of the food industry or the pharmaceutical industry.

The invention is explained in more detail in the following drawings. However, the invention is by no means limited to the embodiments described therein. Rather, combinations of individual characteristic of embodiment are also possible and included in the basic idea of the invention.

### BRIEF DESCRIPTION OF THE DRAWINGS

FIG. 1 shows a side view of a first embodiment of a microfluidic cell according to the invention.

FIG. 2 shows a top view of the embodiment shown in FIG. 1.

FIG. 3 shows a side view of another embodiment.

FIG. 4 shows a top view of the embodiment of FIG. 3.

FIG. 5 shows the both embodiments of FIG. 1/2 and FIG. 3/4 from the front side.

FIG. 6 shows an exploded drawing of an embodiment of a flow cell with cover plate and bottom.

FIG. 7 shows an embodiment with removable cover plate and fixed bottom.

FIG. 8 shows an embodiment with removable cover plate and removable bottom.

FIG. 9 shows an arrangement of several support plates connected in a row.

FIG. 10 shows a holding device (frame) with two integrated flow cells.

FIG. 11 shows individual components of a holding device in form of a frame.

FIG. 12 shows a system for analyzing or diagnosing biofilms and cell cultures, consisting of a stand, holding device and rotary joints for pivoting one or more flow cells.

FIG. 13 shows an advanced embodiment with control elements.

FIG. 14 shows a block diagram of a device controlled via microcontroller.

### WAYS TO CARRY OUT THE INVENTION

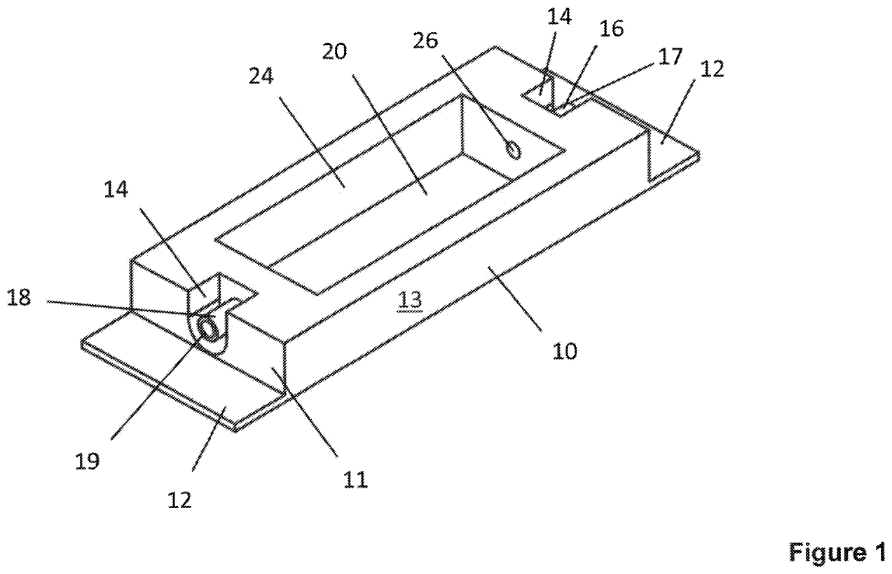

FIG. 1 shows a first embodiment of a microfluidic flow cell according to the invention. This comprises a support plate 10 with a sample chamber 20 formed therein, which is peripherally limited by four chamber walls 24. An inlet 16 with an integrated inlet channel 17 and a drain 18 with an integrated drain channel 19 can be seen on the front sides 11 of the support plate 10. The inlet channel 17 or the drain channel 19 lead into the sample chamber 20 via corresponding openings 26 in the chamber wall 24. In the embodiment displayed, the inlet 16 and the drain 18 are embedded in a slot 14 in the support plate 10. The slot facilitates the connection of the pipes to the inlet 16 or from the drain 18 and their stability without disturbing the external geometry of the cell.

To ensure compatibility with normal and inverted microscopes, holding elements 12 can be found on the front sides 11 of the support plate 10, in order to attach the support plate 10 to a microscope stage or alternatively to a holding device (e.g., for vertical operation in a 90° position). This holding device preferably allows an adjustment within an angular range of 0° to 180°, facilitating the colonization of biofilms on both sides of the surfaces. In the embodiment displayed, the holding elements 12 are strip-shaped. i.e. designed as a rectangular web, and preferably have an aspect ratio between the long side and the short side of 3:1. The width of the holding elements 12 are preferably equivalent with the width of the support plate 10. In the embodiment displayed, the long side of the holding elements 12 (i.e., the front side 11 of the support plate 10) is 24 mm long, while the holding elements 12 have a width of 8 mm. The overall length of the support plate 10, including the two holding elements 12, is 75 mm with a width of approximately 24 to 25 mm. Thus, the flow cell according to the invention differs only slightly from the measurements of a conventional microscopy stage, making it compatible with various microscopes. The two holding elements 12 allow the cell to be easily attached to the microscope stage. Preferably, the depth of the sample chamber is between 7 and 8 mm, making the cell compatible with both normal and inverted microscopes. In the preferred embodiment, the sample chamber 20 itself is 40 mm long and 16 mm wide.

According to the invention, in order to lower the pressure within the sample chamber 20, the diameters of the inlet channel 17 of the inlet 16 and of the drain channel 19 of the drain 18 are extended, in particular to a diameter larger than 1 mm, preferably larger than 1.5 mm.

The embodiment displayed is designed without a bottom 22 (not shown), but can be equipped with a removable or permanently fixed bottom 22, if required. A cover plate 40 is placed on the top of the support plate 10 and connected to the support plate 10. This provides a sealed sample chamber 20. If the cover plate 40 is transparent, the experimental procedure and the individual parameters can be easily observed. In order to pass large flow rates into the enlarged sample chamber 20 and to prevent connecting tubes from sliding to the inlet 16 or drain 18, the length of the inlet 16 or drain 18 is about 5 mm. This allows large flow rates of up to 100 mi/min.

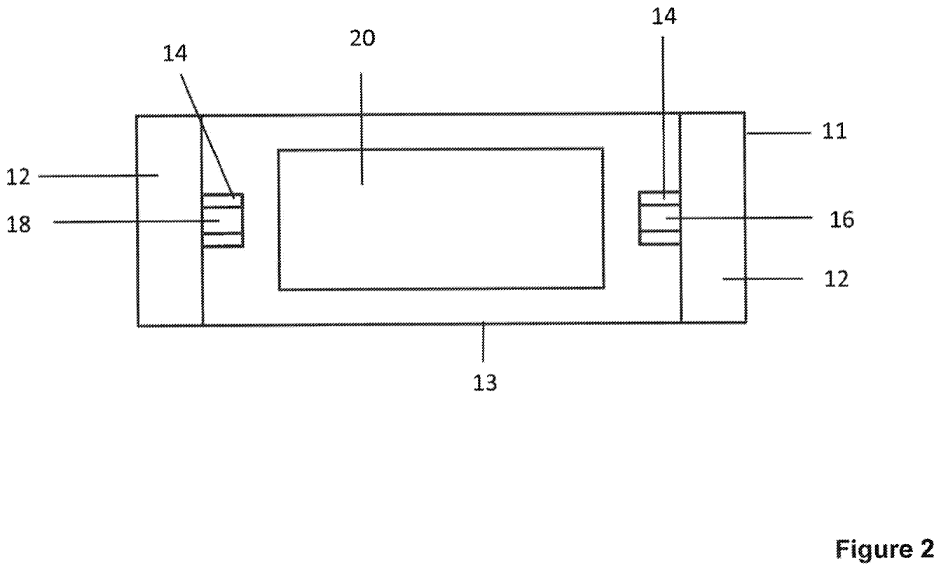

FIG. 2 shows a top view of the embodiment displayed in FIG. 1. The dimensions of the support plate 10, the holding elements 12 and the sample chamber 20 can be seen.

In all embodiments, the length ratio of the front sides 11 to the longitudinal side 13 including holding elements 12 is about 1:3. Lengths of the inlets 16 and the drains 18 between 5 mm and 10 mm are preferred. Preferred diameters of the inlet channel 17 of the inlets 16 and the drain channel 19 of the drains 18 are between 1.5 mm and 2.5 mm.

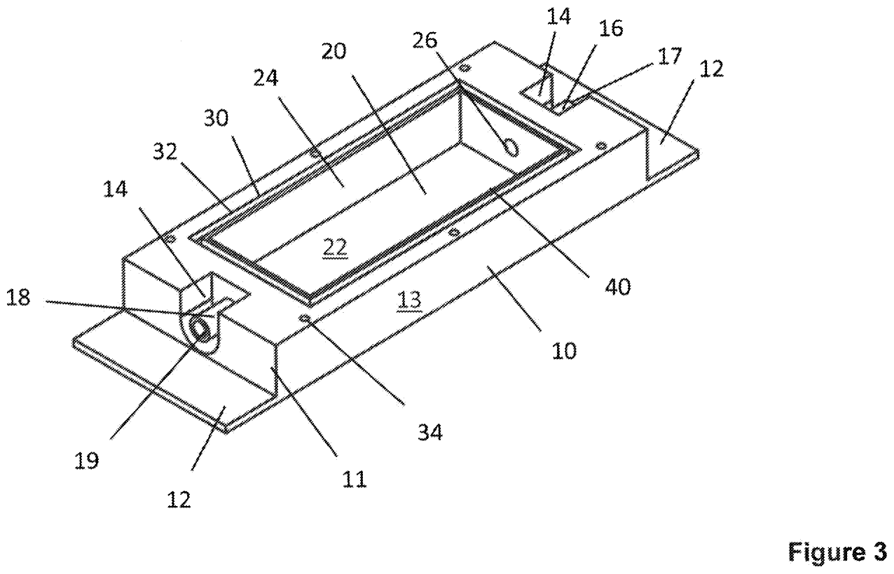

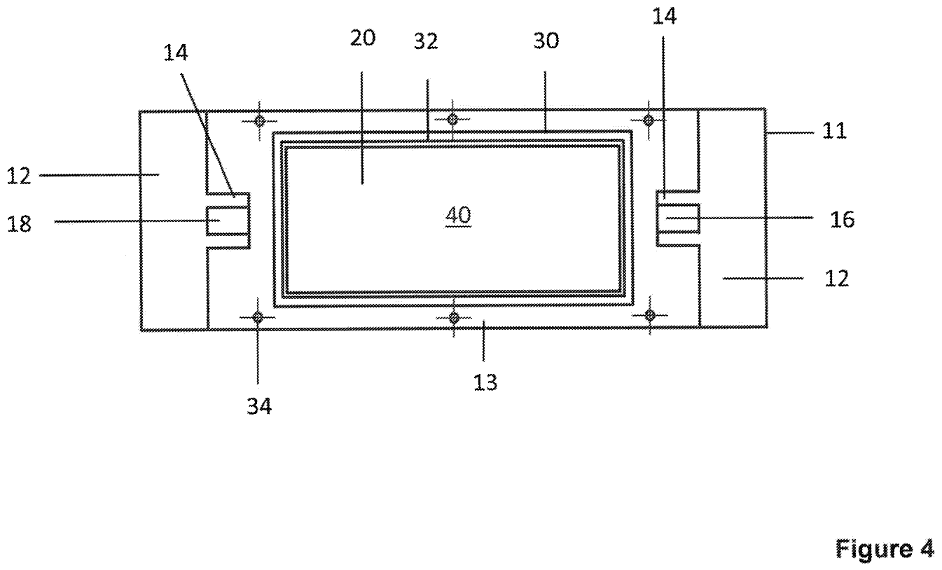

FIG. 3 shows a further embodiment in which the sample chamber 20 can be equipped either with or without a bottom 22. The special feature of this embodiment is a circumferential notch 30 on top of the support plate 10 for a seal 32. In the embodiment in which the bottom plate is can be removed, there is also a notch 30 on bottom of the support plate 10 for liquid-tight sealing of the cell. The bottom 22 is either tightly attached to the support plate 10 or, depending on the embodiment, is designed as a removable bottom plate. The seal 32 externally seals the sample chamber 20. On top of the support plate 10, mounting holes 34 can be seen, to which a separate see-through window can be attached. The embodiment displayed can be seen again in FIG. 4 from a top view. The cover plate 40 is preferably transparent. The choice of materials for the cover plate 40 or the bottom 22 can create ideal experimental conditions for analyzing biofilms or cell cultures, in particular for performing macroscopic analyses of biological samples.

Since the height of the sample chamber 20 is preferably between 8 and 10 mm, it is possible to operate at a significantly reduced pressure, which protects the biofilms to be examined and also averts any possibly occurring air bubbles. In addition, the height of the sample chamber 20 makes it possible to operate in a vertical mode, so that the biofilm can be cultivated on both surfaces of the cover plate 40 and the bottom 22. This does not require an air bubble trap, unlike conventional solutions. The holding elements 12 on the front sides 11 of the support plate 10 allow alternating between a horizontal, vertical or stepless setup in an angular range between 0° and 180°. Another advantage of the geometry according to the invention is that several flow cells can be inserted into each other in a modular manner, i.e., a space-saving grouping is possible.

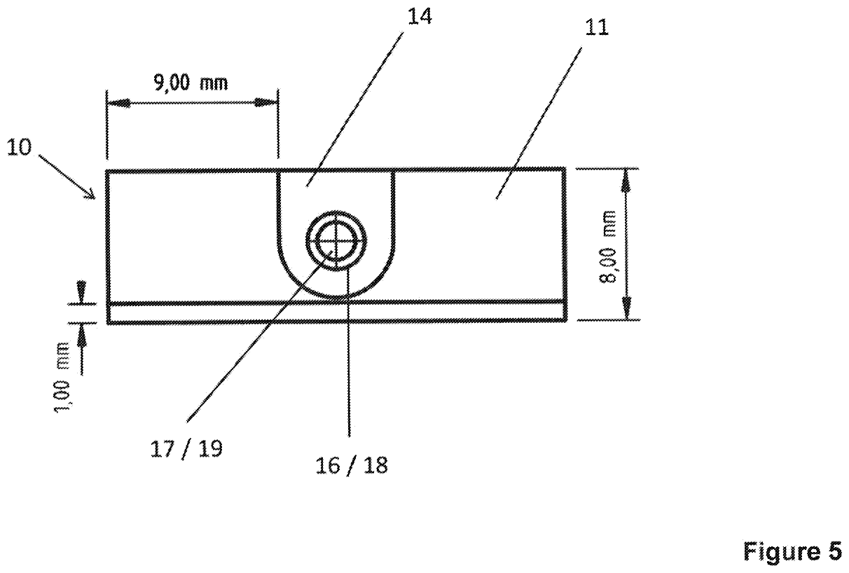

FIG. 5 shows a side view of the front sides 11 of the support plate 10 of the two embodiments described above. The U-shaped slot 14 with the enclosed inlet 16 or outlet 18 and the integrated inlet channel 17 or drain channel 19 can be seen.

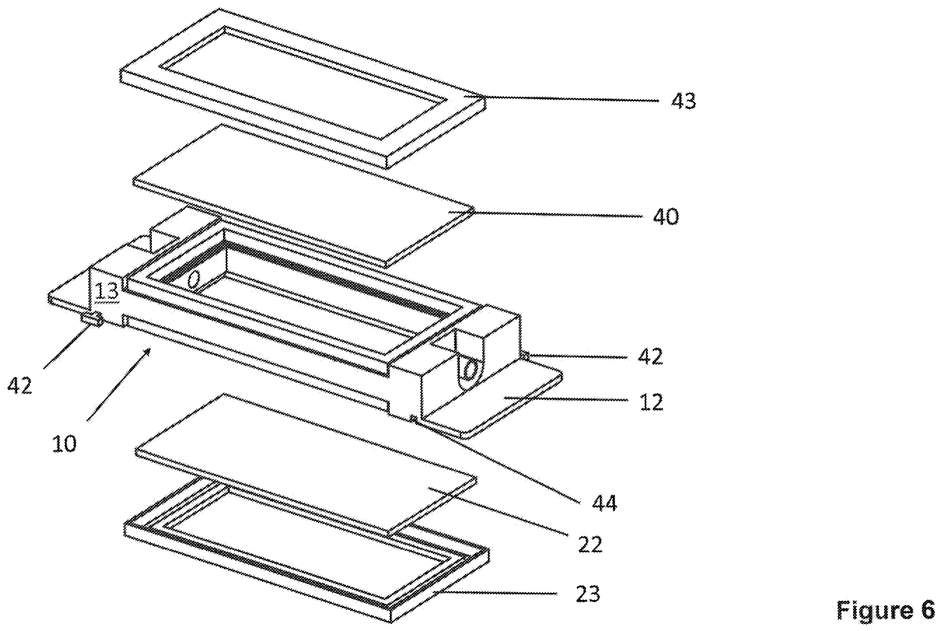

FIG. 6 shows an embodiment of the flow cell according to the invention in which the cover plate 40 is held by a cover frame 43. The bottom 22 in turn is held by a bottom frame 23. In this embodiment, both surfaces, i.e., the cover plate 40 or the bottom 22, can be exchanged. In doing so, different surfaces can be colonized with biofilms or cell cultures using a flow cell. The biological samples can be used for further experiments outside the flow cell. This enables, for example, the use of special antibodies, fluorescent markers or DNA probes for analyzing or diagnosing, where tiniest amounts of the substances have to be applied to the surface with precise accuracy. At least one latching lug 42 is also attached on the longitudinal side 13 of the support plate 10, which cooperates with a corresponding latching receptacle 44 of an adjacent flow cell. In the embodiment displayed, a latching lug 42 and a latching receptacle 44 can be found on each longitudinal sides 13 of the support plate 10. Corresponding characteristics can also be found on the opposite sides, but shifted so that in case of a serial setup of individual flow cells, the adjacent support plates 10 can each be connected to one another in a modular manner.

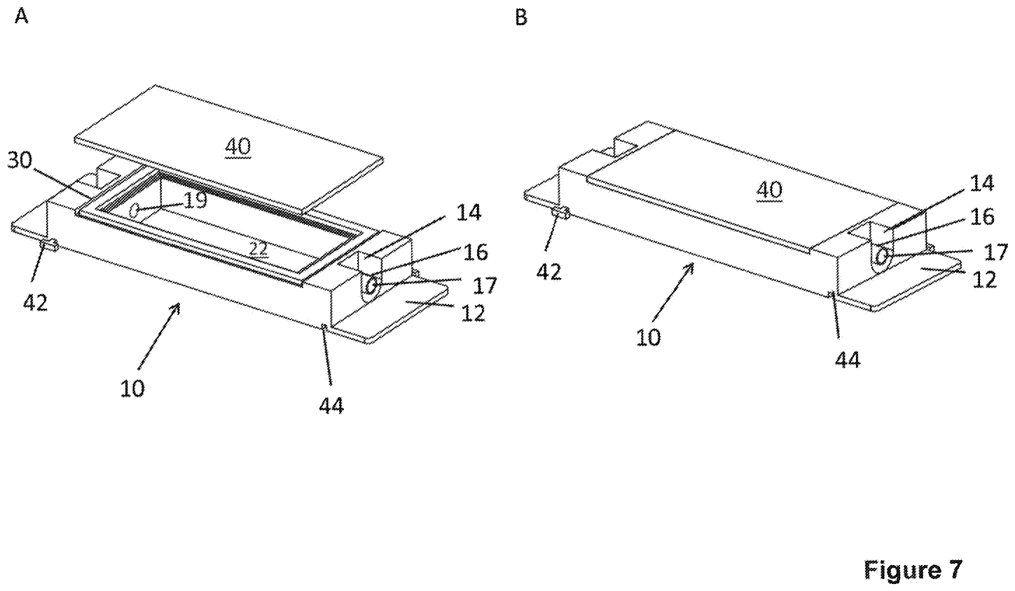

FIG. 7 shows the embodiment in which only the cover plate 40, but not the bottom 22, is removable. Here, the latching lugs 42 and latching receptacles 44 can be seen, too. FIG. 7A illustrates that the cover plate 40 is removable while the bottom 22 is fixed. FIG. 7B shows the combined design. This embodiment is suitable for applications that do not involve microscopy. For example, macroscopic analyses of biological samples can be performed with this embodiment. Experiments can be observed live through a transparent cover plate 40. For follow-up experiments, the samples can be removed from the sample chamber 20 via the cover plate 40.

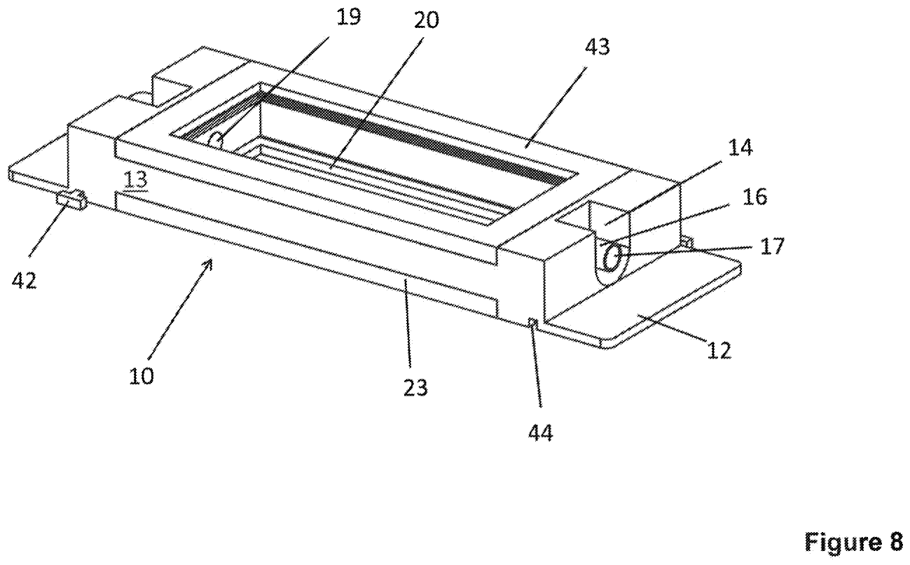

FIG. 8 shows the embodiment with removable cover plate 40 and bottom 22 (not shown). Here, the latching lugs 42 and the latching receptacles 44 can be seen, too. Furthermore, the removable cover frame 43 for holding the cover plate 40 and the bottom frame 23 for holding the bottom 22 can also be seen. The cover frame 43 and the bottom frame 23 are designed in a way that they can incorporate the surfaces to be colonized with the biofilm or the cell cultures, i.e., the cover plate 40 or the bottom 22.

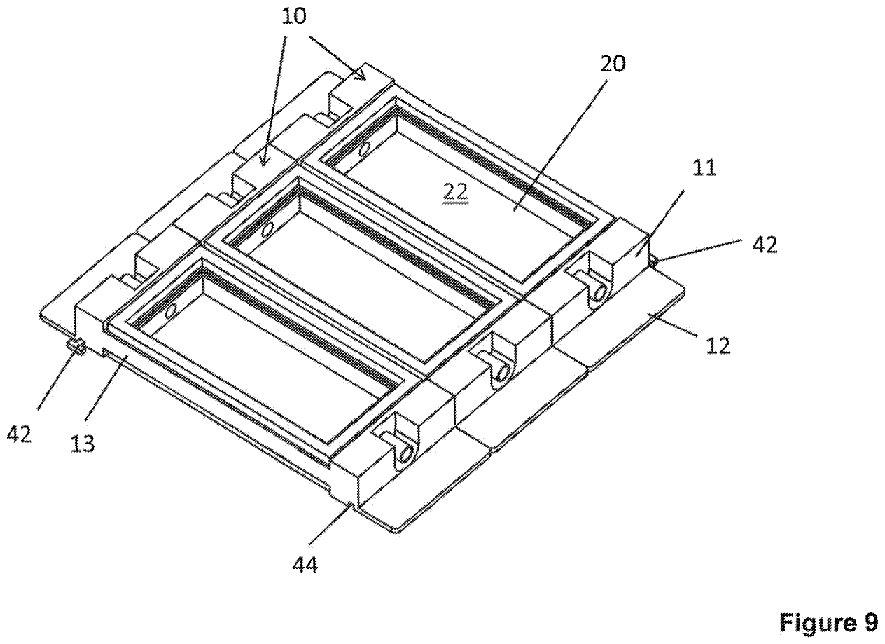

FIG. 9 shows the modular arrangement of three flow cells. The individual support plates 10 are connected to each other via the latching lugs 42 or latching receptacles 44 attached to the longitudinal sides 13.

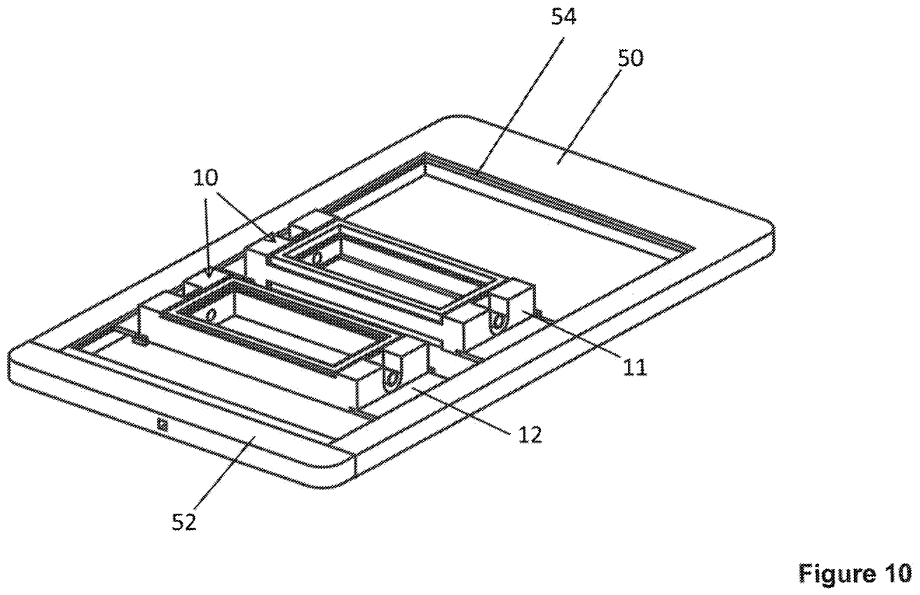

FIG. 10 shows a holding device 50 according to the invention, which consists of a frame with several frame elements. In the embodiment displayed, a frame element 52 is placed on another frame element of the frame, closing the frame circumferentially. The support plates 10 of the two flow cells displayed lead into the notch 54 of the frame via the holding elements 12 on the front sides 11, and are thereby held within the holding device 50.

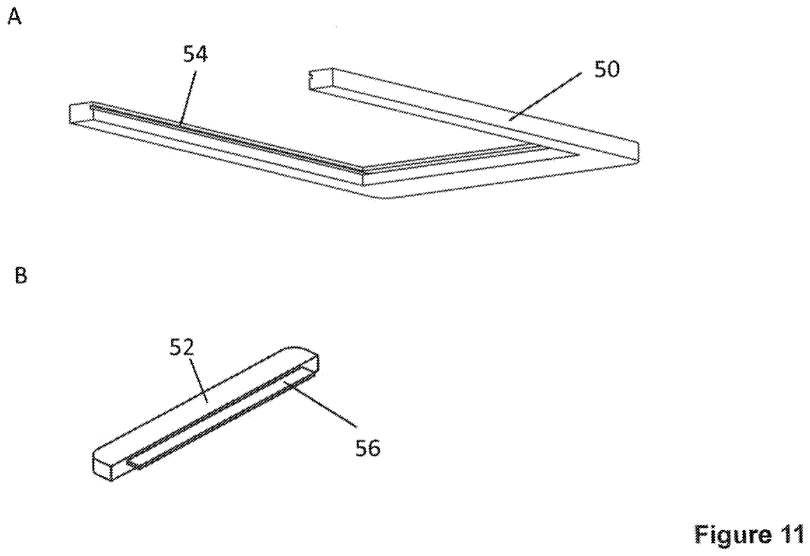

FIG. 11 again displays the individual frame elements in detail. The frame element 52 (FIG. 11B) is attached to the frame of the holding device 50 (FIG. 11A) via a corresponding lug 56, whereby the lug 56 of the one frame element (52) cooperates with the notch 54 of the frame.

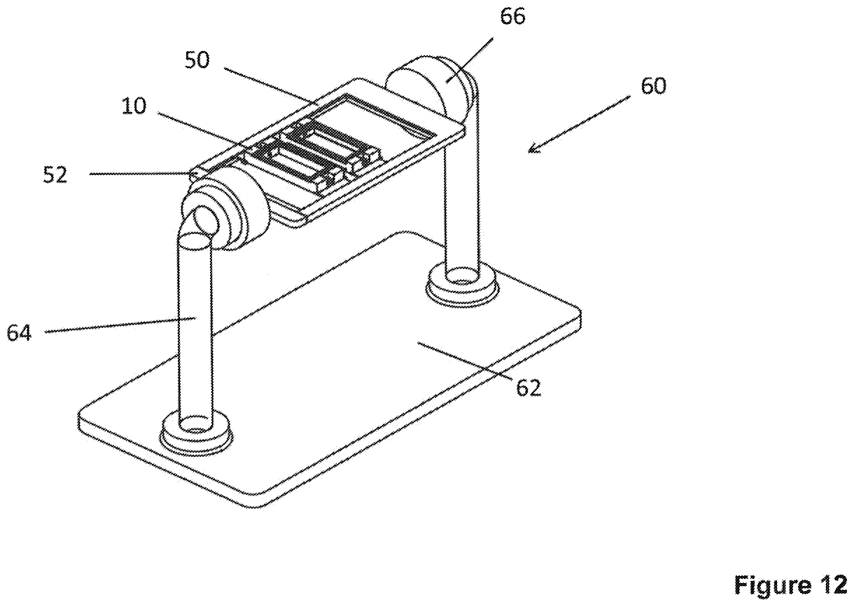

FIG. 12 shows a system for analyzing or diagnosing biofilms and cell cultures, comprising a stand 60, a stand plate 62, supporting elements 64 and rotary joints 66, between which a holding device 50 according to the invention with the support plates 10 arranged therein is attached in a pivotable manner. This system allows multiple flow cells to be stored together in a pivotable manner, as the frame of the holding device 50 can accommodate multiple flow cells. The rotary joints 66 enable the entire holding device 50 with the flow cells received therein to rotate over an angular range of preferably 180°. Alternatively, a stepper motor can be used (not shown). The flow cell is connected to a pump via appropriate lines and connections enabling the inlet 16 and drain 18 to fluidically communicate with each other.

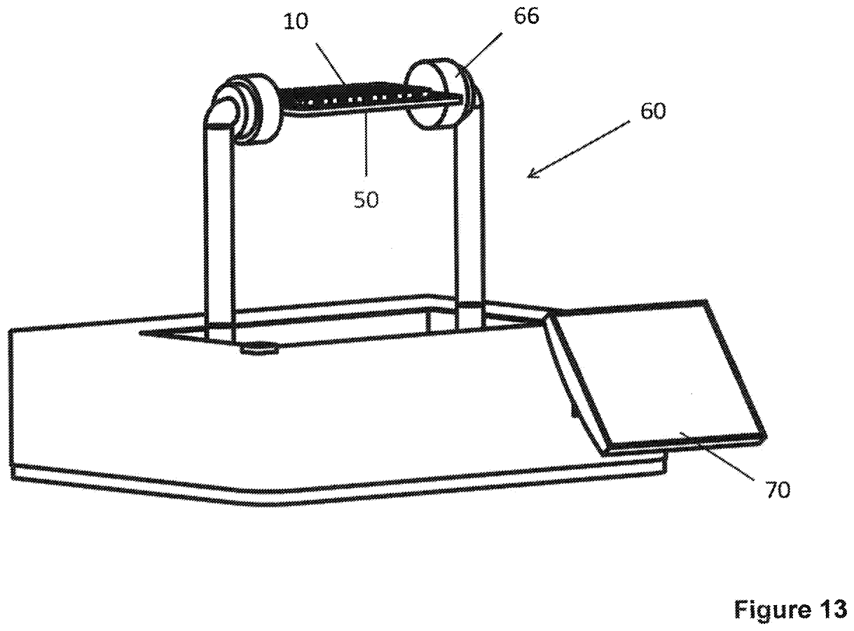

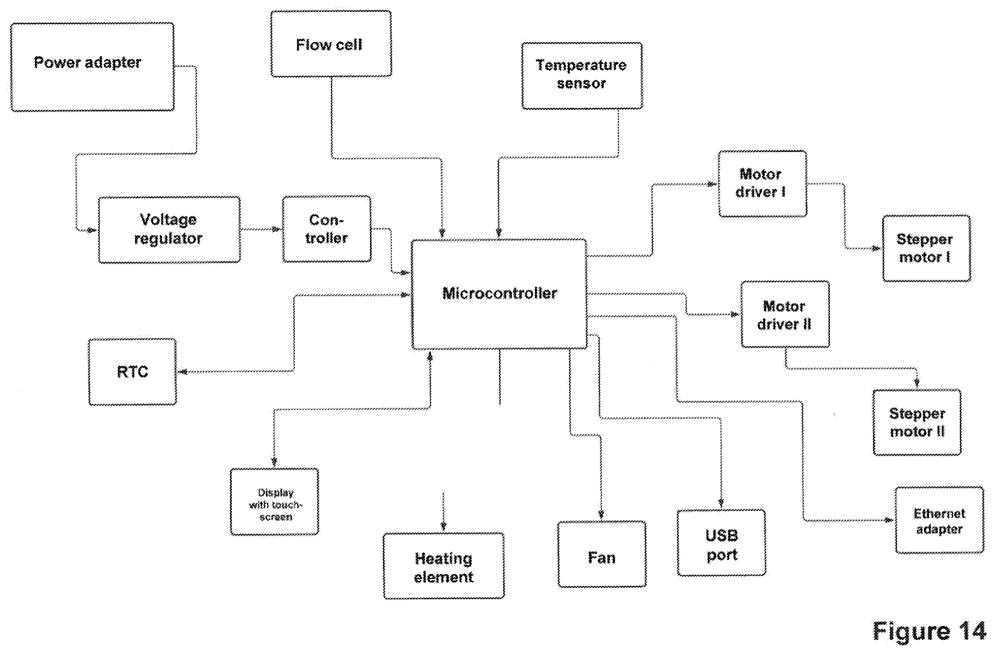

FIG. 13 shows a system for the analyzing or diagnosing biofilms and cell cultures, which is equipped with one or more microfluidic flow cells. A holding device 50 incorporates a support plate 10 with a sample chamber 20 formed therein. Via rotary joints 66, the support plate 10 is pivotable within an angular range between 0° and 180°, preferably an operating range between 0 and 90° is selected. Via supporting elements 64, the holding device 50 is connected to the system. The system comprises a microcontroller (not shown) and is equipped with corresponding sensors in order to receive parameters required for analysis or diagnostics and to perform corresponding control. Thus, the system includes at least one flow sensor and one temperature sensor to determine the flow rate or temperature in the sample chamber. The microcontroller then ensures that the aforementioned parameters are achieved via corresponding control systems. For example, the system includes a heating element and a fan to provide predetermined incubation conditions. Further, the microcontroller also drives one or more drive units, for example stepper motors, to lead an incubation medium into the sample chamber and to pivot the support plate 10 within a predetermined angular range. A display with touchscreen 70 for monitoring and control can be found on the front and be used for programming. The control and regulation units of the system according to the invention are explained in more detail in FIG. 14.

FIG. 14 shows a block diagram of a system controlled by a microcontroller for the analyzing or diagnosing biofilms and cell cultures. The key feature of the system is a microcontroller that performs various tasks. A power adapter and a voltage regulator as well as appropriate controlling elements are required to operate the system. A real time clock (RTC) is responsible for the timing and sequence of individual process steps, i.e., the RTC is used to determine when and for how long certain process steps are to be carried out. Programming can be carried out according to previously defined parameters. A temperature sensor is responsible for the monitoring of the temperature and ensures the transmission of the data to the microcontroller. If the temperature falls below a certain, previously defined threshold, a heating element is activated via the microcontroller so that the temperature is increased. Vice versa, if the temperature in the sample chamber or the temperature of the incubation medium is above a certain threshold, the microcontroller controls a fan. The microcontroller also controls the drive units, i.e., the motor drivers and the stepper motors. Motor driver I and stepper motor I ensure that the incubation medium is lead into the sample chamber. Motor driver II and stepper motor II ensure that the support plate is rotated within a previously defined angular range or to a defined angle. In doing so, the microcontroller can be programmed to gradually change the angle within a period of time. For example, in the adhesion phase, the angle can be 0°, with the plate continuously pivoted up to 90° over time. In this angular range, air bubbles will escape upwards so that the surface of the slide is bubble-free. For connectivity, the system is further equipped, for example, with a USB port and an Internet adapter. A display with touchscreen ensures corresponding operability by the user.

### REFERENCES

AbuZineh K, Joudeh L I, Al Alwan B, Hamdan S M, Merzaban J S, Habuchi S (2018). Microfluidics-based super-resolution microscopy enables nanoscopic characterization of blood stem cell rolling. Sci Adv. 4(7):eaat5304. doi: 10.1126/sciadv.aat5304.
Alessandri, K., u.a.: All-in-one 3D printed microscopy chamber for multidimensional imaging, the UniverSlide. Sci.Rep. (2017) 7:42378.
Babic J, Griscom L, Cramer J. Coudreuse D (2018). An easy-to-build and re-usable microfluidic system for live-cell imaging. BMC Cell Biol. 19(1):8. doi: 10.1186/s12860-018-0158-z.
Bryers J D (2008). Medical biofilms. Biotechnol Bioeng. 1; 100(1):1-18. doi:10.1002/bit.21838.
de Carvalho C (2018). Marine Biofilms: A successful microbial strategy with economic implications. Frontiers in Marine Science. 5. 10.3389/fmars.2018.00126.
Epshteyn, A. A., u.a.: Membrane-integrated microfluidic device for high-resolution live cell imaging. Biomicrofluidics (2011) 5(4): 046501-046501-6.
Flemming H C, Wingender J, Szewzyk U, Steinberg P, Rice S A, Kjelleberg S (2016). Biofilms: an emergent form of bacterial life. Nat Rev Microbiol. 11; 14(9):563-75. doi: 10.1038/nrmicro.2016.94.
Fleming D, Rumbaugh K P (2017). Approaches to Dispersing Medical Biofilms. Microorganisms. 1; 5(2). pii: E15. doi: 10.3390/microorganisms5020015.
Gale B, Jafek A, Lambert C, Goenner B, Moghimifam H, Nze U, Kamarapu S (2018). A review of current methods in microfluidic device fabrication and future commercialization prospects. Inventions. 3.60. 10.3390/inventions3030060.
Hall-Stoodley L, Costerton J W, Stoodley P (2004). Bacterial biofilms: from the natural environment to infectious diseases. Nat Rev Microbiol. 2(2):95-108.
Little B, Lee J, Ray, R (2008). The influence of marine biofilms on corrosion: A concise review. Electrochimica Acta. 54. 2-7. 10.1016/j.electacta.2008.02.071.
Niaounakis M (2017). Degradation of plastics in the marine environment. 10.1016/B978-0-323-44354-8.00003-3.
Paie P, Martinez Vázquez R, Osellame R, Bragheri F, Bassi A (2018). Microfluidic Based Optical Microscopes on Chip. Cytometry A. 93(10):987-996. doi:10.1002/cyto.a.23589.
Pinto M, Langer T M, Hüffer T, Hofmann T, Hemdl G J (2019). The composition of bacterial communities associated with plastic biofilms differs between different polymers and stages of biofilm succession. PLoS One. 14(6):e0217165. doi: 10.1371/journal.pone.0217165.
Sakai K, Chariot F, Le Saux T, Bonhomme S. Nogue F. Palauqui J C. Fattaccioli J (2019). Design of a comprehensive microfluidic and microscopic toolbox for the ultra-wide spatio-temporal study of plant protoplasts development and physiology. Plant Methods. 15:79. doi: 10.1186/s13007-019-0459-z.
Tolker-Nielsen T, Steinberg C (2014). Methods for studying biofilm formation: flow cells and confocal laser scanning microscopy. Methods Mol Biol. 1149:615-29. doi: 10.1007/978-1-4939-0473-0_47.
Wolfaardt G M, Lawrence J R, Robarts R D, Caldwell S J, Caldwell D E (1994). Multicellular organization in a degradative biofilm community. Appl Environ Microbiol. 60(2):434-46.

1. A microfluidic flow cell for analyzing or diagnosing biofilms and cell cultures, comprising:
a support plate (10) with a sample chamber (20) formed therein, which is peripherally limited by chamber walls (24) and a bottom (22),
a cover plate (40), which can be connected with the support plate (10) in a fluid tight manner,
an inlet (16) with an integrated inlet channel (17), which leads to the sample chamber (20) via an opening (26),
a drain (18) with an integrated drain channel (19),
characterized by the following features:
holding elements (12) for fixing the support plate (10) to a microscope stage or a holding device (50) are attached to the front sides (11) of the support plate (10).

2. The microfluidic flow cell according to claim 1, characterized in that the cover plate (40) and/or the bottom (22) are attached the support plate (10) in a removable manner.

3. The microfluidic flow cell according to claim 1, characterized in that the holding elements (12) arranged on the front sides (11) of the support plate (10) of the flow cell are strip-shaped, whereas the aspect ratio between the long side and short side is preferably 3:1.

4. The microfluidic flow cell according to claim 1, characterized in that a latching lug (42) and a latching receptacle (44) each are attached to at least one longitudinal side (13) of the support plate, which cooperate with the corresponding latching receptacles (44) and latching lugs (42) of adjacent flow cells.

5. The microfluidic flow cell according to claim 1, characterized in that the diameters of the inlet channel (17) of the inlet (16) and the drain channel (19) of the drain (18) are >1 mm.

6. The microfluidic flow cell according to claim 1, characterized in that a notch (30) for attaching a seal (32) is formed on the top and bottom of the support plate (10).

7. The microfluidic flow cell according to claim 1, characterized in that the cover plate (40) is incorporated in a cover frame (43) and/or the bottom (22) is incorporated in a bottom frame (23).

8. The microfluidic flow cell according to claim 1, characterized in that at least the support plate (10) is entirely made out of polyethylene (PE) or polypropylene (PP).

9. A system for analyzing or diagnosing biofilms and cell cultures, comprising:
one or more microfluidic flow cells according to claim 1,
a holding device (50) for holding one or more flow cells, with the holding device (50) being designed in a way that one or more flow cells can be pivoted within an angular range of 0° to 180°,
a pump, which is fluidically connected to the inlet (16) and the drain (18).

10. The system according to claim 9, characterized in that the holding device (50) is a frame with several frame elements (52), with the frame comprising a notch (54) for receiving one or more flow cells, which is cooperating with the holding elements (12) of the support plate (10).

11. The system according to claim 9, characterized in that the holding device (50) is a frame with several frame elements (52), with the frame comprising a notch (54) for receiving one or more flow cells, which is cooperating with the holding elements (12) of the support plate (10).

12. The system according to claim 9, characterized in that the holding device (50) with one or more flow cells can be pivoted within an angular range of 0° to 180° via rotary joint or a stepper motor.

13. The system according to claim 9, characterized in that the flow sensor is configured to determine the flow rate of the incubation medium within the sample chamber (20).

14. The system according to claim 9, characterized in that the temperature sensor is configured to determine the temperature within the sample chamber (20).

15. The system according to claim 9, characterized in that a microcontroller controls the regulation of the flow rate, the temperature, the angular range or the inflow of the incubation medium.

16. The system according to claim 15, characterized in that the microcontroller controls a heating element or fan for temperature control.

17. Use of a microfluidic flow cell according to claim 1 or of a system according to claim 9 for analyzing and diagnosing biofilms or cell cultures.

18. The system according to claim 9 characterized in that the one or no low cells can be pivoted within an angular range of 0° to 90°.
